(19) United States
(12) Patent Application Publication
(10) Pub. No.: US 2004/0101900 A1
(43) Pub. Date: May 27, 2004

(54) Assay for rapid detection of TNT

(76) Inventors: J. Mattew Mauro, Silver Spring, MD (US); Ellen R. Goldman, Germantown, MD (US); George P. Anderson, Seabrook, MD (US); Phan T. Winter, Silver Spring, MD (US)

Correspondence Address: NAVAL RESEARCH LABORATORY, ASSOCIATE COUNSEL (PATENTS), CODE 1008.2, 4555 OVERLOOK AVENUE, S.W., WASHINGTON, DC, 20375-5320, US

(21) Appl. No.: 10/303,174

(22) Filed: Nov. 25, 2002

(51) Int. Cl. G01N 33/53

(52) U.S. Cl. 435/7.1; 435/6; 436/518; 436/172

(57) ABSTRACT

An assay to detect and quantitate TNT and compounds related to TNT in aqueous solutions, including seawater, containing a TNT analog bound to a TNT recognition element wherein there is a measurable property change, such as a decrease in TNT analog fluorescence emission intensity, when a test sample containing TNT or related compounds is added to the assay causing free TNT to displace the TNT analog from the TNT recognition element. Also disclosed is the related method of detecting and quantitating TNT and compounds related to TNT.

### BACKGROUND

1. Field of the Invention

The present invention relates to assays and, more specifically, to assays for the detection of 2,4,6-trinitrotoluene (TNT) and compounds related to TNT.

2. Description of the Prior Art

The clean up of areas previously used for munitions storage and processing has generated a need for rapid, selective, and reliable methods for on-site quantification of explosives, including TNT. Environmental monitoring has focused on identifying areas in soil, groundwater, and seawater contaminated with TNT from past munitions manufacture, storage, disposal, and leakage from unexploded ordnance. On-site analysis methods can be used to assess the nature and extent of contamination as well as to monitor remediation progress. A problem with current on-site methods, such as ELISA (enzyme-linked immunosorbent assay), HPLC, GC, MS, and sensor devices, is that they often involve multiple steps and require between 20 minutes and two hours. More rapid methods for dissolved TNT analysis exist, such as continuous flow immunoassays, which have been integrated into field-portable units. The continuous flow immunoassay is a displacement method that measures down stream fluorescently labeled antigen (fluorescent analog) that dissociates from an antibody bound on a solid support due to the addition of antigen to the flow stream (U.S. Pat. No 5,183,740, issued Feb. 2, 1993 to Ligler et al.). The amount of fluorescently labeled antigen released (and subsequently measured down stream of the solid support) is proportional to the amount of antigen added. In continuous flow assays, it is necessary to move by physical solvent flow the released fluorescently labeled antigen away from undisplaced labeled antigen remaining bound on the solid support in order to fluorimetrically quantify the amount of fluorescent material released as a result of up stream antigen addition. Although down stream measurement of the released fluorescent antigen analog in continuous flow assays can provide an analytical result in only a few minutes, it is a serial method and, therefore, suffers from significant inherent and unavoidable sample throughput limitations. In addition, currently available continuous flow immunoassay methods are unable to provide highly sensitive and rapid detection of TNT present in seawater.

### SUMMARY

The aforementioned problems are overcome by the present invention wherein the presence of TNT in aqueous solutions, including seawater, can be detected in high throughput parallel formats by means of an assay system containing a TNT analog bound to a TNT recognition element (such as an anti-TNT antibody) and monitoring a property change, such as TNT analog fluorescence emission intensity, when free TNT added to the assay system displaces the TNT analog from the TNT recognition element. No washing or flow separation steps are required when using this method. Quantitative measurement of TNT added to the assay system results from the change in a property of the labeled TNT analog, such as fluorescence emission intensity, due only to its differential physical behavior under the micro-environmental conditions inherent to its bound versus its free state.

The present invention results in several advantages. Unlike the current methods for detecting TNT, the assay of the present invention is fast (on the order of 5 minutes or less for 96 plus assays), reasonably sensitive, simple to perform—technically trained operators are not needed, easily adaptable to high-throughput formats utilizing parallel sample processing, amenable to automation, and inexpensive—hundreds of assays could be performed for just pennies apiece. In addition, the assay of the present invention is able to detect TNT at levels less than 1 ng/ml in 100% seawater.

### BRIEF DESCRIPTION OF THE DRAWINGS

These and other objects, features and advantages of the invention, as well as the invention itself, will become better understood by reference to the following detailed description, appended claims, and accompanying drawings where:

FIG. 1 shows the fluorescence signal response from the addition of the anti-TNT antibody in Example 1;

FIG. 2 shows the fluorescence signal response from the addition of TNT in Example 2;

FIG. 3a shows the change in fluorescence for the homogeneous plate-based assay using the A1.1.1 anti-TNT antibody in Example 3;

FIG. 3b shows the change in fluorescence for the homogeneous plate-based assay using the 11B3 anti-TNT antibody in Example 3;

FIG. 4 compares the homogeneous and adsorbed assay formats for the detection of TNT in artificial seawater;

FIG. 5 shows the cross-reactivity data for compounds structurally related to TNT and explosives other than TNT; and

FIG. 6 shows the change in fluorescence for the adsorbed format plate based assay in Example 4.

### DETAILED DESCRIPTION

The assay of the present invention can be used to detect explosives such as TNT and related compounds in aqueous solutions based on property changes, such as fluorescence, phosphorescence, polarization, or fluorescent excited state lifetime. The property change can have either a positive or a negative signal. In a preferred embodiment, the assay is based on the fact that there is an increase in fluorescence when a TNT recognition element (e.g. an anti-TNT antibody) binds to a fluorescent TNT analog. TNT can then be detected and quantitated by measuring the change in fluorescence that occurs when unlabeled TNT competes with the fluorescent TNT analog for binding to the anti-TNT antibody binding site. The fluorescence decreases as the free TNT displaces the fluorescent TNT analog from the TNT recognition element. The assay can be adapted to high throughput formats. Additionally, the assay can be performed on both cuvette- and plate-based formats. With the plate-based format, the antibody can either be bound to the plate or free in solution (i.e. a homogeneous assay).

The assay can be used as either an indicator or a quantitator of TNT. As an indicator, the fact that the fluorescence level decreases signifies that TNT is present. The amount of TNT present can be quantified by measuring how much the fluorescence has decreased.

The assay of the present invention can be adapted to a micro-array format. It can be readily adapted to detect TNT derivatives and TNT breakdown products using the same fluorescent analog. In addition, antibodies for alternative small molecules can be used where there is an appropriate fluorescent analog.

To prepare the assay, start with the TNT analog. The TNT analog can be any molecule that undergoes a measurable change in an observable property when bound or released from a TNT recognition element. In a preferred embodiment, the fluorescent TNT analog is cyanine diaminopentane trinitrophenyl (Cy5-DAP-TNP), which can be prepared as described in A. W. Kusterbeck, P. Charles, C. Patterson, P. Gauger, Curr. Protoc. Field Anal. Chem., 2000, 2C11.1-2C11.14, incorporated herein by reference. Then add the TNT recognition element, which can be any TNT binding molecule, causing the TNT analog to bind to the TNT recognition element. Examples of TNT binding molecules include antibodies, recombinant antibody fragments, peptides, peptide nucleic acids, nucleic acid aptamers, molecular imprinted surfaces, and other polymer types. In a preferred embodiment, the TNT recognition element is monoclonal antibody A1.1.1 (made by Strategic Biosolutions) or 11 B3 (made by Intracell) with A1.1.1 being preferred. Next add the sample to be tested and measure the change in fluorescence as TNT in the test sample competes for binding with the TNT analog. The order of addition of the TNT analog, the anti-TNT antibody, and the test sample is not critical and can be done in any order.

### EXAMPLE 1

### Demonstration of Fluorescence Emission Change Upon Anti-TNT Antibody/Cy5-DAP-TNP Complex Formation

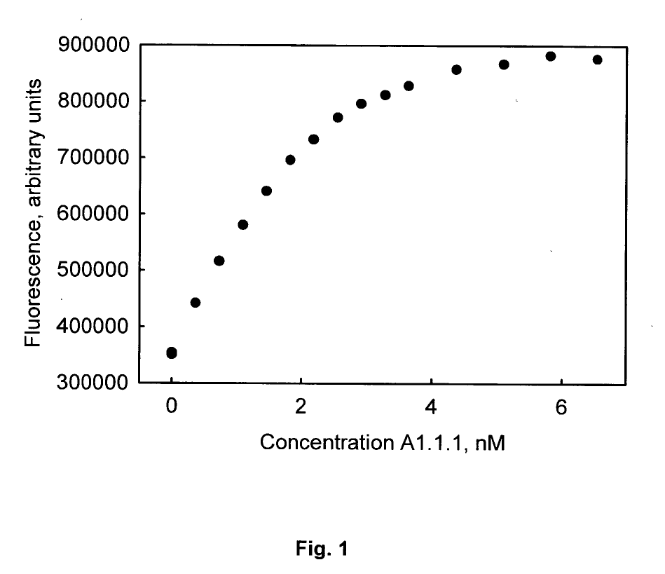

Anti-TNT antibody/Cy5-DAP-TNP complex formation was studied using a SPEX Fluorog-2 fluorimeter, with excitation at 600 nm and right angle emission observed at 662 nm with 15 nm slit widths. All measurements were made on samples prepared using phosphate buffered saline (PBS; 2 ml) at 22° C. Beginning with the fluorescent Cy5-DAP-TNP ligand in the cuvette at a concentration of 5 nM, anti-TNT antibody A1.1.1 was added in 15 increments over a total range of 0.36 nM to 6.54 nM of antibody. The total volume of antibody stock solution added was 30 μl, or about 2% of the initial volume. When increasing amounts of A1.1.1 (from 0.36 nM to 6.54 nM) were added to a cuvette containing 5 nM Cy5-DAP-TNP and the fluorescence recorded, an initial steep rise in fluorescence on addition of antibody from 0.36 nM to about 3 nM occurred, followed by a plateau at around 5 nM with about a three-fold increase in fluorescence intensity observed over the course of the titration (see FIG. 1). This type of fluorescence change may be due to environmental, electronic and conformational effects that occur in the Cy5-DAP-TNP analog upon binding to the antibody.

### EXAMPLE 2

### Homogenous Competitive Fluorimmuno Titration with TNT

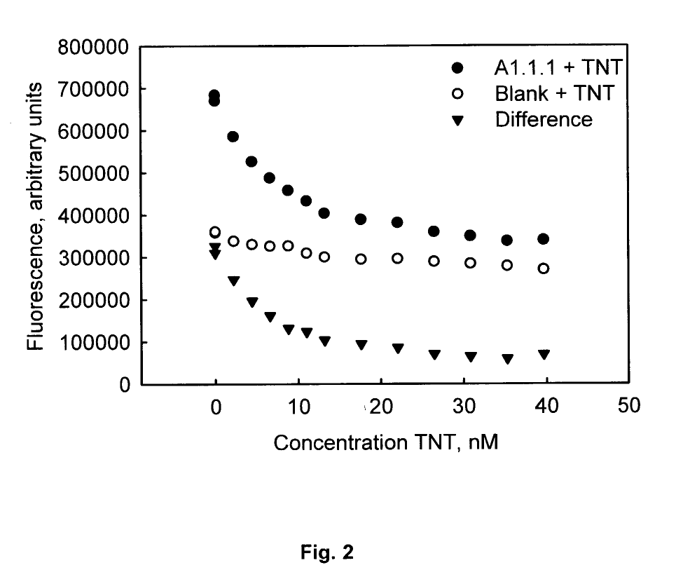

For TNT analysis, a cuvette containing 5 nM Cy5-DAP-TNP was prepared and anti-TNT antibody A1.1.1 was added to 1.1 nM, a concentration in a region of high slope on the antibody/Cy5-DAP-TNP binding curve under these conditions. Aqueous TNT solution was then added in increments over a total range from 0.5 ng/ml up to 9 ng/ml and the decrease in fluorescence was recorded after each addition (see FIG. 2). The total volume of added TNT solution was 18 μl, or about 1% of the initial volume. An identical control experiment was performed in which PBS buffer was substituted for A1.1.1 antibody solution. This blank titration data was subtracted from the assay values to construct a final corrected titration curve. Fluorescence signal changes from the blank titration were minimal relative to the sample with antibody.

### EXAMPLE 3

### Microtiter Plate-Based Homogenous Competitive Fluoroimmunoassays

Wells of white 96-well plates (microfluor 2, Dynex) were blocked with a solution of 1% bovine serum albumin (BSA) in Tris buffered saline (50 mM Tris-HCL, 105 mM NaCl, pH 7.5; TBS) for at least 2 hours. The blocking solution was removed and 0.33 pmol of anti-TNT antibody (A1.1.1 or 11B3) in 25 μl PBS was added to each of a series of wells followed by 2.5 pmol Cy5-DAP-TNP (in 25 μl PBS). Next, 50 μl solutions of TNT (in PBS) were added to test wells to final concentrations spanning 0.05 ng/ml to 10,000 ng/ml. Controls with 50 μl PBS in place of TNT solution were also performed. The fluorescence was read in a SpectraFluor Plus microtiter plate reader (Tecan) using a 620 nm excitation filter (10 nm band pass) and a 670 nm (25 nm band pass) emission filter. Control wells containing no antibody were typically included in each run. Fluorescence data were background corrected by taking the difference between emission values obtained for wells with TNT and control wells to which only PBS buffer was added.

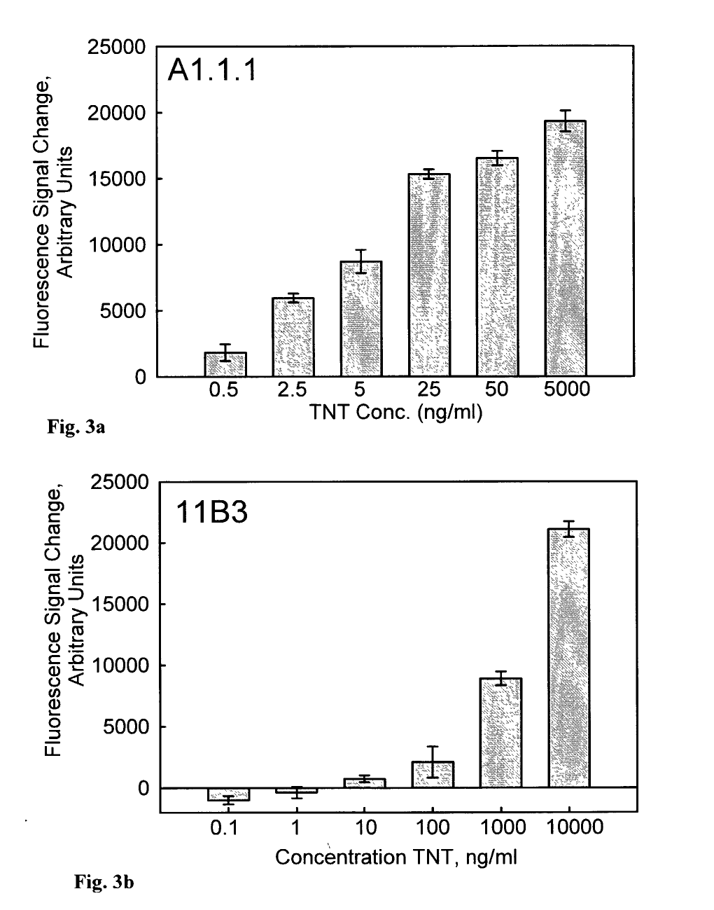

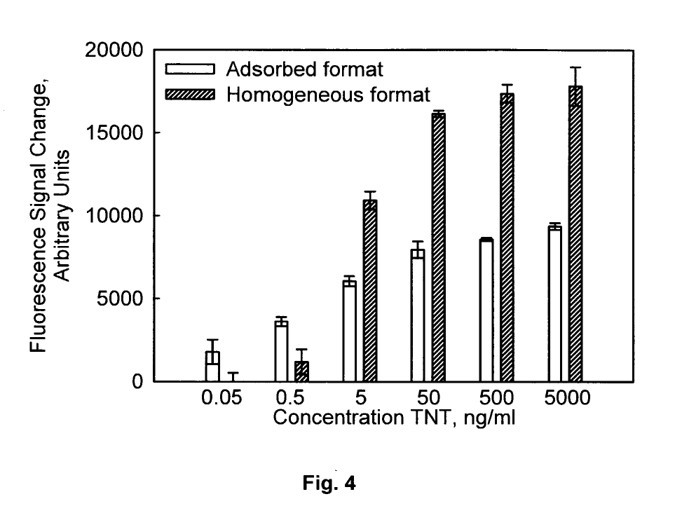

As was seen in the cuvette-based experiments, the plate-based homogeneous assays showed an increase in fluorescence (about two-fold) on the addition of Cy5-DAP-TNP to antibody (A1.1.1 or 11B3). Fluorescent analog bound to both anti-TNT antibodies A1.1.1 and 11B3 showed a systematic decrease in fluorescence on the addition of increasing amounts of TNT. A range of TNT concentrations spanning 0.05 ng/ml through 10,000 ng/ml was examined. Using the A1.1.1 and 11B3 antibodies, TNT was detected in PBS with limits of detection of 0.5 ng/ml and 10 ng/ml respectively (see FIGS. 3a and 3b). The difference in limits of detection probably reflects the fact that the 11B3 antibody does not have as high an affinity for TNT as the A1.1.1 anti-TNT antibody, a fact that has been demonstrated previously in different types of assays comparing the antibodies. A1.1.1 antibody was also used in homogeneous assays performed in artificial seawater (prepared from dry material using deionized water according to the manufacturer's directions (Sigma)); these assays gave a limit of detection of 0.5 ng/ml (see FIG. 4). The assay worked well in the high ionic strength environment eliminating the need for dilution or extraction when testing for TNT in seawater.

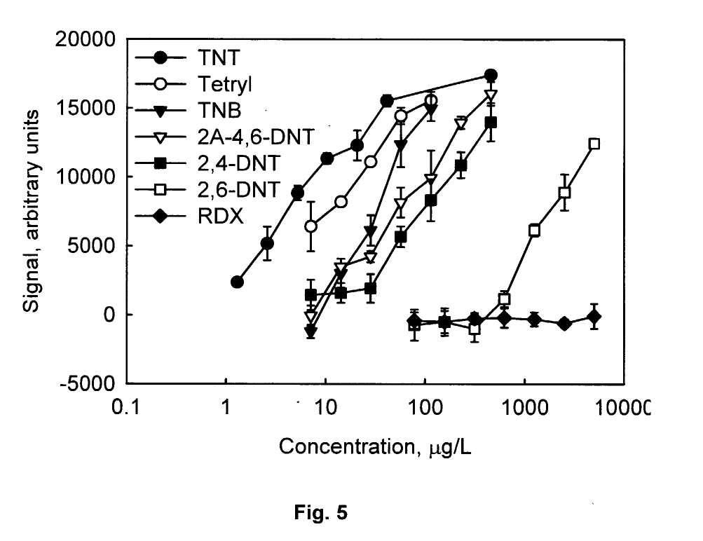

Plate-based homogeneous assays were also performed with artificial seawater substituted for PBS as the assay medium. Additionally, plate-based homogenous cross reactivity assays were carried out in PBS under identical conditions using known TNT breakdown products, structurally related nitroaromatic compounds, or other nitro based explosives in place of TNT to test assay specificity. The TNT related compounds 2-amino-4,6-dinitrotoluene (2A-4,6-DNT), 2,4-dinitrotoluene (2,4-DNT), 2,6-dinitrotoluene (2,6-DNT), 1,3,5-trinitrobenzene (1,3,5-TNB), and 2,4,6-trinitrophenyl-n-methylnitramine (tetryl), and the explosive hexahydro-1,3,5-trinitro-1,3,5-triazine (RDX) were examined. Concentrations between 7 and 5000 μg/L were used to construct a curve for each of the compounds (see FIG. 5). Percent cross-reactivities, with Tetryl cross-reacting the most (45%), followed by TNB (19%), 2A-4,6-DNT (9%), 2,4-DNT (5%) and 2,6-DNT (0.3%) with RDX showing no cross-reactivity were calculated from the concentrations corresponding to half of the maximum signal.

### EXAMPLE 4

### Solid Phase Microtiter Plate Based Competitive Fluoroimmunoassays

Wells of the white 96-well plates were coated with 10 μg/ml of the anti-TNT antibody A1.1.1 diluted in 0.1 M NaHCO₃ (pH 8.6) by adding 100 μl per well and incubating overnight at 4° C. The next day, the antibody solution was removed and the plates blocked with 1% BSA in TBS. A 25 nM solution of Cy5-DAP-TNP in PBS was added to each well and allowed to bind for approximately 15 minutes. The Cy5-DAP-TNP solution was then discarded, the wells washed with PBS and solutions of TNT (diluted in PBS) from 0.05 to 10,000 ng/ml were added to the wells. The fluorescence was then measured in the plate reader as previously described. Data was plotted as the difference between the fluorescence in wells with added TNT and the wells to which only buffer was added.

This assay format was also expanded to include artificial seawater as the assay medium. Additionally, TNT breakdown products or other explosives were substituted for the TNT in order to examine the specificity of the assay.

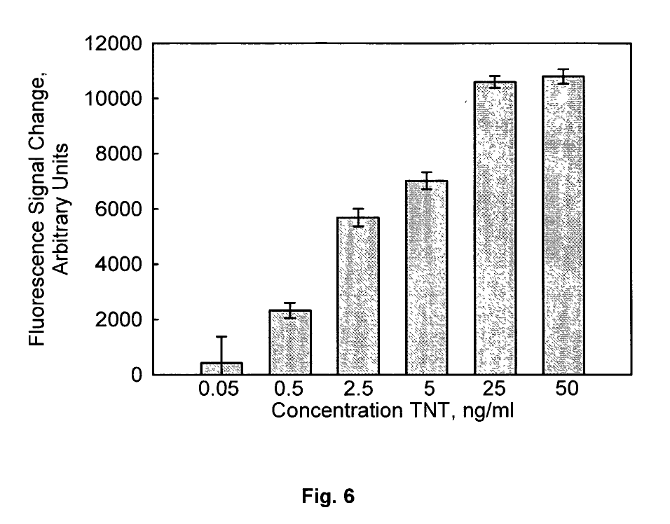

An alternative plate-based version of the assay in which antibody was adsorbed to the wells instead of used in solution was developed in which Cy5-DAP-TNP was bound to plate-immobilized antibody and excess fluorescent reagent discarded before the addition of TNT spiked solutions. A1.1.1 antibody was utilized for detecting TNT in both PBS (see FIG. 6) and artificial seawater (see FIG. 4) in the antibody adsorbed configuration and yielded limits of detection of 0.5 ng/ml and 0.05 ng/ml respectively.

### EXAMPLE 5

### Specificity of Antibody—TNT Interaction

Six compounds, either structurally related to TNT or other explosives were examined in both plate assay formats to explore antibody specificity in these formats. The A1.1.1 antibody exhibited basically the same cross reactivity trend as reported in the following references: Anne Zeck, M. G. Weller & Reinhard Niessner, Characterization of a monoclonal TNT-antibody by measurement of the cross-reactivities of nitroaromatic compounds, FRESENIUS' J. ANAL. CHEM., Vol. 364, Issue 1/2, pp. 113-120 (1999); and Upvan Narang, Paul R. Gauger & Frances S. Ligler, A Displacement Flow Immunosensor for Explosive Detection Using Microcapillaries, Anal. Chem., Vol. 69, pp. 2779-2785 (1997), both of which are incorporated herein by reference. Representative data for the homogeneous format are shown in FIG. 5, with tetryl cross-reacting the most, followed by 1,3,5-TNB, 2A-4,6-DNT, and 2,4-DNT with both 2,6-DNT and RDX showing no cross reactivity within the error of the assay.

### EXAMPLE 6

### Comparison of the Two Methods for Plate Based Assays

Both of the plate-based formats examined have advantages. The homogeneous assay is fast, requires only a blocked plate, involves no wash steps, and requires only a few minutes incubation time prior to reading. The adsorbed assay is more sensitive (in assays performed in artificial seawater), but it requires more preparation time. Plates must be coated with antibody, blocked, loaded with Cy5-DAP-TNP and washed prior to TNT addition. Potentially, antibody-coated and blocked plates could be prepared in advance and stored. Storage of plates loaded with the dye analog would make this assay format potentially as fast as the homogeneous assay. However, Cy5-DAP-TNP is prone to degradation when stored in solution or kept at room temperature, so fully loaded plates may need to be stored at low temperature.

In these formats the fluorescence intensity is dependent on the exact amount and concentration of the fluorescent probe and is sensitive to small variations in pipetted volume. Automated reagent delivery would be preferable to minimize errors due to test sample and reagent delivery variation.

The above description is that of a preferred embodiment of the invention. Various modifications and variations are possible in light of the above teachings. It is therefore to be understood that, within the scope of the appended claims, the invention may be practiced otherwise than as specifically described. Any reference to claim elements in the singular, e.g. using the articles “a,” “an,” “the,” or “said” is not construed as limiting the element to the singular.

1. An assay to detect and quantitate 2,4,6-trinitrotoluene (TNT) and compounds related to TNT in an aqueous solution without requiring a continuous flow, comprising:
a TNT recognition element; and
a TNT analog bound to the TNT recognition element;
wherein there is a measurable property change of the TNT analog when a test sample containing TNT or compounds related to TNT is added to the assay causing free TNT or related compounds to displace the TNT analog from the TNT recognition element.

2. The assay of claim 1 wherein said TNT recognition element, said TNT analog, and said test sample can be added in any order.

3. The assay of claim 1 wherein said TNT recognition element is any TNT binding molecule.

4. The assay of claim 1 wherein said TNT recognition element is selected from the group consisting of antibodies, recombinant antibody fragments, peptides, peptide nucleic acids, nucleic acid aptamers, molecular imprinted surfaces, other polymer types, and mixtures thereof.

5. The assay of claim 1 wherein said TNT recognition element is selected from the group consisting of monoclonal antibodies A1.1.1 and 11B3 and mixtures thereof.

6. The assay of claim 1 wherein said property change is a change in fluorescence.

7. The assay of claim 1 wherein said TNT analog is cyanine diaminopentane trinitrophenyl (Cy5-DAP-TNP) or other chemical analogs that function similarly.

8. The assay of claim 1 wherein said TNT recognition element is free in solution.

9. The assay of claim 1 wherein said TNT recognition element is bound on a solid surface.

10. The assay of claim 1 wherein said TNT recognition element is bound to a well in a plate.

11. The assay of claim 1 wherein said aqueous solution comprises seawater.

12. A method to detect and quantitate 2,4,6-trinitrotoluene (TNT) and compounds related to TNT in an aqueous solution without requiring a continuous flow, comprising the steps of:
(a) binding a TNT analog to a TNT recognition element;
(b) measuring a property of the TNT analog after it is bound to the TNT recognition element;
(c) combining the TNT analog and the TNT recognition element with a sample to be tested for TNT or compounds related to TNT; and
(d) measuring a change in the property of the TNT analog caused by free TNT or related compounds displacing the TNT analog from the TNT recognition element.

13. The method of claim 12 wherein said TNT recognition element is any TNT binding molecule or surface.

14. The method of claim 12 wherein said TNT recognition element is selected from the group consisting of antibodies, recombinant antibody fragments, peptides, peptide nucleic acids, nucleic acid aptamers, molecular imprinted surfaces, other polymer types, and mixtures thereof.

15. The method of claim 12 wherein said TNT recognition element is selected from the group consisting of monoclonal antibodies A1.1.1 and 11B3 and mixtures thereof.

16. The method of claim 12 wherein said property is fluorescence.

17. The method of claim 12 wherein said TNT analog is cyanine diaminopentane trinitrophenyl (Cy5-DAP-TNP) or other chemical analogs that function similarly.

18. The method of claim 12 wherein said TNT recognition element is free in solution.

19. The method of claim 12 wherein said TNT recognition element is bound on a solid surface.

20. The method of claim 12 wherein said TNT recognition element is bound to a well in a plate.

21. The method of claim 12 wherein said aqueous solution comprises seawater.
